(19) United States
(12) Patent Application Publication
(10) Pub. No.: US 2016/0168521 A1
(43) Pub. Date: Jun. 16, 2016

(54) BIOREACTORS SUPPORTED WITHIN A RACK FRAMEWORK AND METHODS OF CULTIVATING MICROORGANISMS THEREIN

(71) Applicant: ALGABLOOM INTERNATIONAL LTD., Richmond (CA)

(72) Inventor: Soheyl MOTTAHEDEH, Coquitlam (CA)

(21) Appl. No.: 14/569,619

(22) Filed: Dec. 12, 2014

(51) Int. Cl. C12M 1/00; C12M 3/00; C12M 1/12; C12N 1/12

(52) U.S. Cl. CPC C12M 21/02; C12N 1/12; C12M 23/44; C12M 23/48; C12M 23/22; C12M 23/04; C12M 23/26

(57) ABSTRACT

A photobioreactor is provided for culturing phototrophic microorganisms comprising a plurality of reaction chambers composed of a translucent, pliable, water-impermeable material, each of the reaction chambers being an elongate sleeve capable of holding a culture medium, and a modular support structure comprising a framework defining first and second sides and a first and second end, and configured to support a plurality of horizontally oriented, vertically spaced shelves, each of the shelves extending from the first to the second side of the framework and having disposed thereon one of the plurality of reaction chambers. The design of the photobioreactor is modular, which can be expanded as needed, while maintaining the ratio of a high density of reaction chambers-to-small external footprint. The bioreactor can be used to grow single-celled micro-organisms and other small multi-cellular organisms.

### CROSS-REFERENCE TO RELATED APPLICATIONS

Pursuant to 37 CFR§1.78(a)(4), this application claims the benefit of and priority to Canadian Patent Application No. 2,836,218 filed Dec. 13, 2013, the entire content of which is herein incorporated by reference.

### FIELD OF THE INVENTION

The invention relates to systems and methods for cultivating photoautotrophic microorganisms. The invention further relates to a photobioreactor system and method for growing and harvesting algae in a mass production environment.

### BACKGROUND OF THE INVENTION

Microorganisms are very diverse and include all the bacteria, the archaea and almost all of the protozoa. They also include some fungi, algae and certain animals such as rotifers. Many macro animals and plants have juvenile stages, which are also microorganisms. A photoautotrophic microorganism is an organism that is capable of generating its own sustenance from inorganic substances using light as an energy source. As an example, photosynthetic microscopic algae, hereinafter referred to as algae, are photoautotrophs. Algae are unicellular organisms, which produce oxygen by photosynthesis, and may include flagellates, diatoms, and blue-green algae. More than 100,000 species of algae are known.

The current energy crisis has prompted interest in alternative energy, bringing a great deal of attention to the production of algae biofuels. Beyond biofuels, commercial algae farming is also important to medicine, food, chemicals, aquaculture and production of feedstocks. One major obstacle to algae farming is the commercial scale-up for mass culture, temperature control of algae and the high cost associated with such a culture. As a result, during the past decade, much focus has been aimed at the production of algae for commercial purposes. This focus is evidenced by the manifestation of many new industries and uses of algal production.

The vast number of different bioreactor concepts is testimony that the best algal farming bioreactors are still to be found. Most bioreactor designs are not suitable for commercial use due to cost and scale-up problems.

A closer look at the systems disclosed in prior documents people skilled in the art have strongly discouraged suspending or supporting horizontally-oriented structures above ground, particularly carrying heavy loads of liquids over suspended structures. This discouragement has been extended even further when liquids were to be carried and contained in flexible or semi-rigid containers. Objectors have argued that such an undertaking calls for extra support costs, requires additional structural stability or may be subject to environmental risks.

Serpentine Processing: Serial Processing through the Reaction Chambers

One strategy for replicating the effectiveness of a raceway pond within a small footprint is to vertically stack a series of horizontally-oriented reaction chambers such that the liquid media from the higher chamber flows into the one immediately below it usually following a serpentine path. The length of the serpentine path creates the length of the “raceway” for the reaction of the algae as each of the reaction chambers are linked in a series.

In U.S. Pat. No. 8,372,632 Kertz teaches a method and apparatus for sequestering CO₂ using algae comprising a plurality of vertically suspended bioreactors, each bioreactor being translucent and including a flow channel formed by a plurality of baffles. A culture tank contains a suspension of water and at least one algae and includes a plurality of gas jets for introducing a CO₂-containing gas into the suspension. The culture tank is in fluid communication with an inlet in each channel for flowing the suspension through the channel in the presence of light. A pump pumps the suspension into the channel inlet.

In U.S. Pat. No. 8,415,142 Kertz provides a method and apparatus for growing algae for sequestering carbon dioxide and then harvesting the algae including a container for a suspension of algae in a liquid and a bioreactor having a translucent channel in fluid communication with the container to absorb CO₂ and grow the algae. A monitor determines the reaction of the algae in the channel. A separator separates the grown algae from the suspension and an extractor extracts biomaterials from the grown algae.

In U.S. Pat. No. 8,713,850, Seebo describes a bioreactor in the form of an algae growing assembly that comprises a plurality of growing trays vertically stacked together and retained within a transparent housing. Each growing tray is configured to flowingly transport nutrient enriched water to the growing tray positioned immediately beneath it. Each growing tray is composed of a stiff transparent plastic sheet having a pliable transparent gas permeable membrane affixed thereon. A carbon dioxide gas infusion system is fluidly connected to each of the plurality of growing trays such that carbon dioxide gas is able to (1) inflate respective carbon dioxide gas chambers, and (2) diffuse into the nutrient enriched water.

Vertical Spacing of the Trays/Shelves Allows for Exposure to Natural Light

There are a series of photo-bioreactor patent documents relating to the arrangement of the vertically stacked growing trays that allows for maximal exposure to natural sunlight.

The photobioreactor disclosed by Levin in US Patent Application No. 2007/0155006 teaches the construction of a photobioreactor, which is based on application of parallel sets of multi-level troughs intended for flowing a microalgae suspension, these troughs are irradiated therewith by the sun light. The troughs are arranged in each set one above the other. The width of the gaps between the neighboring sets of the troughs is significantly larger than the width of the troughs themselves. Optical elements, which reflect and disperse the light, are positioned between the neighboring sets of the troughs. The broth with microalgae is flowing from the troughs into a collecting troughs and thereupon this suspension is accumulated in a tank. The suspension is supplied again from the tank by a pumping means into the feeding pipes and thereafter—to the troughs.

In US Patent Application No 2011/0300614, Tian Kian Wee teaches a photobioreactor comprising a base; a supportive frame extending upwardly from the base; a plurality of trays for culturing phototropic microorganisms, ranking vertically from a uppermost tray to a bottommost tray, supported co-axially on the supportive frame and spaced apart one and other in a predetermined gap at the vertical plane to optimize exposure to a light source; and a protective member located on top of the uppermost tray by mounting onto the supportive frame wherein the plurality of trays and the protective member are made of light permeable material.

Primarily Defined by Natural Light Sources, Supplemented by Artificial Light Sources

Rusiniak in U.S. Pat. No. 8,800,202 discloses a biomass production apparatus comprising a stack of trays, each tray, in use, being in receipt of a respective layer of liquid, the layers being spaced apart from one another such that each layer has associated therewith a respective headspace. Light sources are provided for each layer and are disposed in the headspace associated with said each layer, to illuminate, at least in part, said each layer.

### SUMMARY OF THE INVENTION

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

This invention relates to a photobioreactor that is constructed by assembling a modular framework and shelves to create a rack structure, which supports the reaction chambers, thereby creating a column of easily accessible reaction chambers. The reaction chambers are constructed from translucent pliable material (such as plastic), enabling the walls to be gusseted into delivery tubes that can be used as sparger tubes or for the delivery of nutrients. Each chamber comprises a nutrient and gas delivery system which also functions as a fluid agitation system. Each reaction chamber is operatively associated with an illumination system and a harvesting system. The design of the photobioreactor is modular, which can be expanded as needed, while maintaining the ratio of a high density of reaction chambers-to-small external footprint. The bioreactor can be used to grow single-celled micro-organisms and other small multi-cellular organisms.

More specifically this invention relates to a photobioreactor for culturing phototrophic microorganisms comprising: a plurality of reaction chambers composed of a translucent, pliable, water-impermeable material, each of the reaction chambers being an elongate sleeve also called plastic film tube capable of holding a culture medium, and a modular support structure comprising a framework defining first and second sides and a first and second end, and configured to support a plurality of horizontally oriented, vertically spaced shelves, each of the shelves extending from the first to the second side of the framework and having disposed thereon one of the plurality of reaction chambers.

The foregoing has outlined rather broadly certain features of the present invention in order that the detailed description of the invention that follows may be better understood. Additional features of the invention will be described hereinafter that form the subject of the claims. It should be appreciated by those skilled in the art that the conception and the specific embodiments disclosed may be readily utilized as a basis for modifying or designing other structures for carrying out the same purposes as the disclosed bioreactor. It should also be realized by those skilled in the art that such equivalent constructions do not depart from the spirit and scope of the invention as set forth in the appended claims.

### BRIEF DESCRIPTION OF THE DRAWINGS

The accompanying figures incorporated in and forming a part of the specification, illustrate several aspects of the present invention and together with the description serve to explain the principles of the invention.

FIG. 1A is a perspective view of a modular photobioreactor in one embodiment comprising multiple rack framework units joined end to end. FIG. 1B shows an enlarged view of the area circled in FIG. 1A, illustrating the details of the front end of this embodiment of the photobioreactor.

FIG. 2 is a cross sectional view of three reaction chambers in one embodiment attached to the left side of the framework and sitting atop flexible shelves.

FIG. 3 is a cross sectional view of two reaction chambers in one embodiment with a thermal regulation chambers positioned underneath each reaction chamber.

FIG. 4 is a cross sectional view of a rigid shelf in one embodiment that creates a channel on one side of a reaction chamber as it conforms to the contour of the shelf.

FIG. 5 is a perspective side view showing the attachment of the shelves within the framework.

FIG. 6 is a perspective view of a photobioreactor in one embodiment with two columns of reaction chambers and at least four framework units have been joined end-to-end to form the length of the photobioreactor.

FIG. 7 is a cross sectional view of the photobioreactor in one embodiment comprising two columns of reaction chambers, wherein the elevational level of the shelves in one column is offset from the shelves supporting the adjacent column.

FIG. 8 is a cross sectional view one embodiment of the photobioreactor wherein the lower half of one reaction chamber is supported by the upper section of the reaction chamber immediately below it.

FIG. 9 a cross section of one embodiment of the photobioreactor as described in FIG. 8, depicting the arrangement of eight reaction chambers.

FIG. 10A is a perspective view of one embodiment of the photobioreactor. FIG. 10B shows an enlarged view of the area circled in FIG. 10A, illustrating the means of connecting the flexible material to the sides of the photobioreactor.

FIG. 11A is a perspective view of one embodiment of the photobioreactor. FIG. 11B shows an enlarged view of the area circled in FIG. 11A, illustrating the means of connecting the flexible material to the sides of the photobioreactor.

FIG. 12A is a perspective view of the cross section of a reaction chamber in one embodiment comprising one nutrient delivery tube and one sparger tube. FIG. 12B shows an enlarged view of the area circled in FIG. 11A, illustrating a cross-sectional view of the sparger tube in one embodiment emitting gas in opposite directions.

FIG. 13 is a perspective view of a method for constructing a delivery tube within a reaction chamber in one embodiment.

FIG. 14 is a perspective view of one embodiment of a method for constructing two delivery tubes within the material forming the bottom wall of a reaction chamber, which will be attached to the upper wall to form the reaction chamber.

FIG. 15 is a cross sectional view of the reaction chamber in one embodiment comprising two sparger tubes.

FIG. 16 is a cross-sectional view of two reaction chambers in one embodiment, wherein each reaction chamber comprises two sparger tubes configured to alternate in the delivery of the gas, thereby causing agitation of the fluid media.

FIG. 17 is a front view multiple flexible light emitting diode mats for illumination of a bioreactor in one embodiment.

FIG. 18 is a cross-sectional view of an oval-shaped light tube in one embodiment.

FIG. 19 is a perspective side view of a reaction chamber in one embodiment comprising round light tubes within the chamber and a harvesting plate positioned to collect the algae surrounding each tube as the plate moves along the length of the light tubes.

FIG. 20 is a perspective view of a close-up of the round light tube and the harvesting plate in one embodiment.

FIG. 21A is a cross-sectional view of a round light tube in one embodiment with an LED situated on a light bar support. FIG. 21B is a cross-sectional view of a flat light tube in one embodiment with an LED situated on a light bar support.

FIG. 22A is a perspective view of a reaction chamber in one embodiment comprising flat light tubes positioned at an angle due to the action of the harvesting plate moving along the flat tubes. FIG. 22B is a perspective view of the reaction chamber in one embodiment as depicted in FIG. 22A, illustrating flat light tubes positioned within a reaction chamber, wherein the light tubes are resting on the bottom of the reaction chamber.

FIG. 23A is a cross-sectional view of a reaction chamber in one embodiment comprising harvesting plate positioned to collect from four flat light tubes. FIG. 23B is a cross-sectional view of the reaction chamber in one embodiment depicted in FIG. 23A, wherein the light tubes are resting on the bottom of the reaction chamber.

FIG. 24 is a cross-sectional side view along the longitudinal axis of a reaction chamber in one embodiment comprising a thermal regulation chamber positioned between the reaction chamber and the shelf with a movable roller separating fluid content in two portions.

FIG. 25 is a cross-sectional side view along the longitudinal axis of a reaction chamber in one embodiment with a movable roller separating fluid content in two portions.

FIG. 26 is a perspective view of the back of a photobioreactor in one embodiment showing several reaction chamber replacement rollers and a bottom conveyor used for harvesting, dewatering and drying.

FIG. 27A is a perspective view of the front of a photobioreactor in one embodiment showing an overflow culture media collection system designed to collect from every other reaction chamber.

FIG. 27B shows an enlarged view of the area circled in FIG. 27A.

FIG. 28 is a perspective view of a photobioreactor in one embodiment protected by a greenhouse cover.

FIG. 29 is a perspective view of a photobioreactor in one embodiment fitted into a shipping container.

### DETAILED DESCRIPTION OF THE INVENTION

A photobioreactor system and method for growing and harvesting photosynthetic organisms is disclosed in various embodiments. The framework of the photobioreactor is modular and hence may be configured to meet a number of different site requirements. Likewise, the system may be reconfigured while in use to accommodate changing needs and conditions. Hence, it is to be understood that the photobioreactor may be implemented in a number of embodiments; and while the photobioreactor will be explained with regard to some specific embodiments, other embodiments are within the scope of the invention and will be readily apparent to those of skill in the art.

However, one skilled in the relevant art will recognize that the various embodiments may be practiced without one or more of the specific details, or with other replacement and/or additional methods, materials, or components. In other instances, well-known structures, materials, or operations are not shown or described in detail to avoid obscuring aspects of various embodiments of the invention.

Similarly, for purposes of explanation, specific numbers, materials, and configurations are set forth in order to provide a thorough understanding of the invention. Nevertheless, the invention may be practiced without specific details. Furthermore, it is understood that the various embodiments shown in the figures are illustrative representations and are not necessarily drawn to scale.

Reference throughout this specification to “one embodiment” or “an embodiment” or variation thereof means that a particular feature, structure, material, or characteristic described in connection with the embodiment is included in at least one embodiment of the invention, but do not denote that they are present in every embodiment. Thus, the appearances of the phrases such as “in one embodiment” or “in an embodiment” in various places throughout this specification are not necessarily referring to the same embodiment of the invention. Furthermore, the particular features, structures, materials, or characteristics may be combined in any suitable manner in one or more embodiments. Nonetheless, it should be appreciated that, contained within the description are features which, notwithstanding the inventive nature of the general concepts being explained, are also of an inventive nature.

This photobioreactor is designed to support the reaction and cultivation of photo-autotrophic microorganisms. The design and use of the photobioreactor will be described and taught using algae as an example. It is to be understood, however, that the photo-bioreactor can be used to cultivate photo-trophic microorganisms and is not to be restricted to just algae. For example, in some situations it may be desirable to cultivate cyanobacteria, which is a phylum of bacteria that obtain their energy through photosynthesis and therefore require a light source.

In some embodiments, a mixotropic culture system is provided wherein the culture may additionally include non-phototrophic microorganisms such as certain forms of bacteria. It may be desirable to culture such non-phototrophic microorganisms either in a mixed culture with phototrophic organisms, or separately wherein some or all of the reaction chambers are designed without a light source, or wherein the light source is simply not turned on. The parallel processing capacity exhibited by the rack support allows for a multiplicity of culturing conditions within the same footprint of floor space.

The Overview of the Photobioreactor

The photobioreactor comprises a modular framework and shelves that create a rack structure, which supports the reaction chambers, thereby creating a column of easily accessible reaction chambers. The reaction chambers are constructed from translucent pliable material (such as plastic), enabling the material, which will form the reaction chamber, to be gusseted to form delivery tubes. The delivery tubes can be used as sparger tubes to diffuse gases in the medium, as delivery tubes for nutrients, or the like. Each reaction chamber comprises nutrient and gas delivery systems. Delivery tubes can also be used as conduits to transmit variations in the gas pressure within the tubes in a manner that results in fluid agitation of the culture media within a reaction chamber. Each reaction chamber is operatively associated with an illumination system and a harvesting system. The design of the bioreactor is modular, which can be expanded or rearranged as needed, while maintaining the ratio of a high density of reaction chambers-to-small external footprint. The bioreactor can be used to grow single-celled micro-organisms and other small multi-cellular organisms.

Providing reaction chambers whose weight is independently supported by shelves which are in turn affixed to the framework eliminates transfer or accumulation of weight or fluid pressure being exercised on lower reaction chamber. This eliminates the need for pumps to operate under higher pressures such as for aerating the bioreactor system 10 or displacing fluids. This saves energy and reduces substantially operation costs.

Certain embodiments of the photobioreactor are shown in FIGS. lA and B wherein a photobioreactor 10 comprises a modular framework 15 and shelves 30 assembled to create a rack structure. The framework 15 comprises opposing first and second sides 11, 12, wherein each side comprises upright vertical support members 16 connected by horizontal longitudinal connecting members 18. The opposing sides 11, 12 are connected by horizontal transverse connecting members 20, which are present at least at each end of the framework resulting in first and second ends 13, 14 to the photobioreactor, and depending on the overall length of the assembled framework, may also be present at intervals along the length of the framework. The shelves 30 are attached between first and second sides 11, 12 of the open framework 15 creating a rack within the framework, which supports pliable reaction chambers 100 constructed of flexible translucent material. As seen in FIG. 2, the reaction chambers 100 supported upon these shelves 30 form a column of reaction chambers 100 when viewed in cross-section or from one of the ends of the bioreactor.

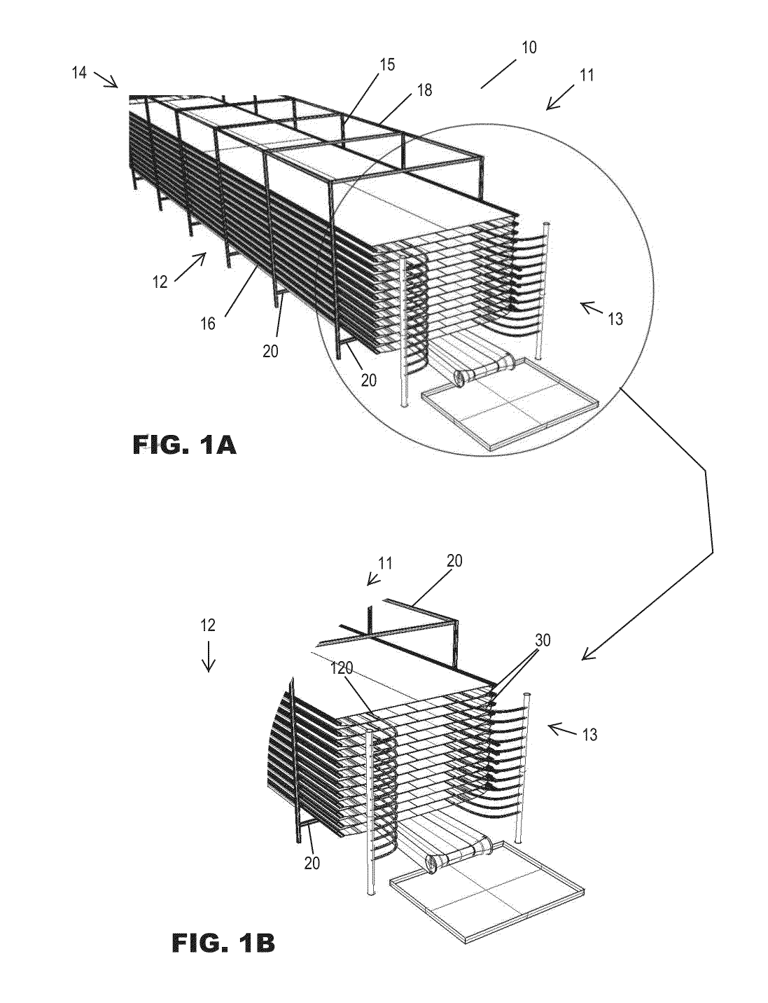

Also seen in FIG. 1, illustrating an embodiment where at least five framework units are connected end-to-end, the shelves 30 in this embodiment extend continuously throughout each level of the framework 15 to support one reaction chamber per level. In alternative embodiments, a series of shorter shelves may be employed which are arranged in spaced relationship along the length of the framework. The amount of spacing between the shelves may vary but is not so extensive that the series of shelves can no longer provide sufficient support for the reaction chamber. Typically, this will mean that each shelf in the series abuts the neighbouring shelf, or that only a small gap is allowed between one shelf and the next in the series. In the embodiment shown in FIG. 1, it can also be seen that neighbouring framework units share upright vertical support members 16.

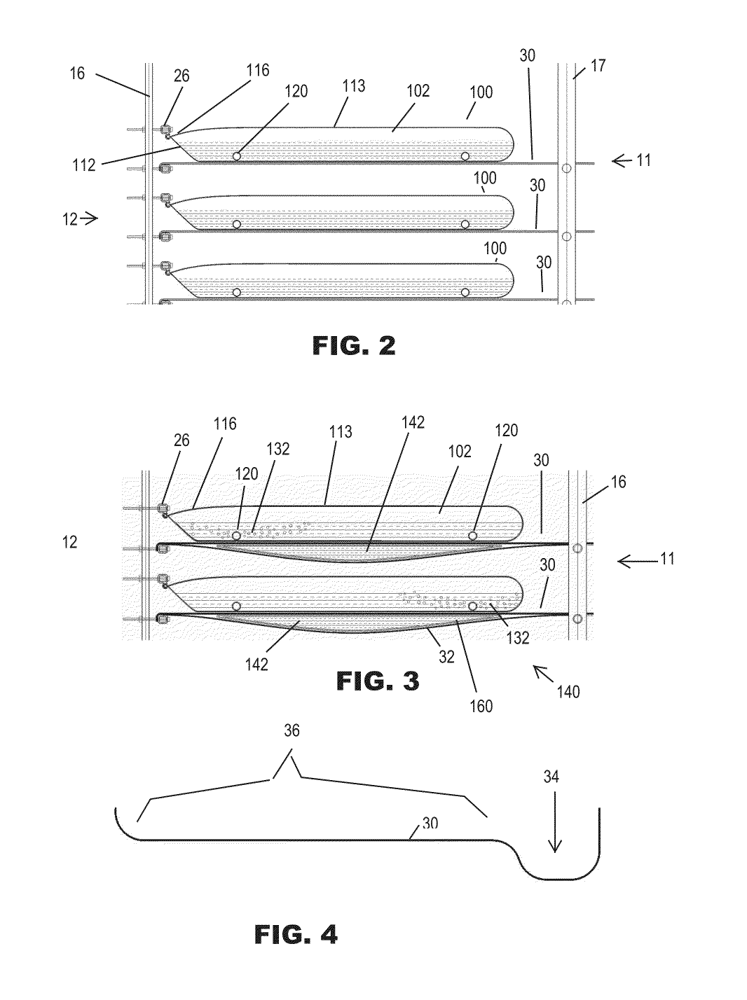

FIG. 2 shows a cross-section of a portion of one embodiment of the photobioreactor illustrating two shelves 30 where flexible material tightly stretched and attached to first and second sides 11, 12 of the framework is used to construct the shelves 30. FIG. 3 shows an embodiment illustrating two shelves 30 wherein tightly stretched flexible material is used to construct the shelves 30. This embodiment also includes a flexible material loosely stretched and connected to the same attachment means as the shelf 30 to create a thermal regulation chamber support 32.

For the purposes of describing the structure of a flow-through photobioreactor, the first end 13 will be considered to be the end of the bioreactor where the inputs such as algae, media, nutrients, and CO₂ are provided to delivery tubes 120 within the reaction chambers 100 and the second end 14 will be considered to be the end of the bioreactor where the algae is harvested. FIG. 2 shows how each reaction chamber is configured to define an interior volume 102, which can retain a culture medium 142 in a suspended culture. Each reaction chamber 100 comprises a nutrient delivery system, a gas delivery system, and a fluid agitation system. Each reaction chamber 100 also has operatively associated with it a lighting system. In one embodiment, the lighting system may be positioned exterior to the reaction chamber. In one embodiment, the lighting system may be an interior lighting system, positioned within the reaction chamber. For example, the lights of the lighting system may be encased in translucent tubular covers 210 running lengthwise throughout the reaction chambers 100.

The multilevel photobioreactor enables an operator to allocate different processes to different reaction chambers 100 located at various levels. The columns of photobioreactor chambers culture the microorganisms in parallel, such that each chamber is isolated from the other chambers avoiding cross-contamination. In one embodiment the photobioreactor can be designed such that the reaction chambers cultivate the algae in serial, whereby the medium from one reaction chamber flows into a second reaction chamber positioned alongside, but at a slightly lower elevation, to the first reaction chamber. This is possible when the embodiment includes two or more columns of reaction chambers.

The bioreactor can be used to grow single-celled organisms and other small multi-cellular organisms. The disclosed bioreactor is generally directed to use for mass culture of algal biomass in a suspended culture being exposed to artificial light, solar light or to a combination thereof.

In one embodiment of the invention, the multilevel bioreactor is contained within a building such as a warehouse, a greenhouse, or contained within a shipping cargo using artificial light such as light emitting diodes LEDs.

The Framework

The framework of the photobioreactor generally defines the outer perimeter of the photobioreactor. It is designed to take into account the production requirements and the space capabilities of the microorganism production facility. The higher the number of shelves stacked vertically above each other in a same framework, the greater the quantity of product that can be produced on the same footprint. The number of shelves is dependent on the overall height of the framework and on the distance provided between the shelves. The inter-shelf distance is limited by the thickness of the shelf support material, the maximum height desired for the medium and by the selected harvesting method. In certain embodiments in which an externally located lighting system is employed, the space needed for the lighting fixture(s) and the effective diffusion of the light into the media will also need to be taken into account when determining an appropriate inter-shelf distance. Experience shows that when LED tapes or LED mats are being used attached the bottom surface of the shelf above, it is possible to position shelves as close as 5 cm apart. However, when using natural light, factors such as shelf width, shadowing effects and light penetration angles must be taken into account. Appropriate inter-shelf distances can be readily determined by the skilled person taking the factors noted above into account. This distance may vary from 5 cm to 25 cm.

The design of the framework of the photobioreactor allows the bioreactor to be expandable as the design is based upon modular framework units that may be arranged in a manner facilitating the efficient deployment and maintenance of the photobioreactor system. The flexible and unique arrangement of the reaction chambers on the horizontal shelves, each being independently secured, and each operating under the same atmospheric pressure at each level of the framework, avoids the vertical build-up of pressure experienced with vertically oriented reaction chambers such as traditional vertical columns, tubes or bioreactor flat-panels.

The selection of materials used to construct the framework will take into account factors such as weight, cost, and space considerations, etc. In some embodiments, it may be preferable to use a material such as stainless steel or powder coated metal, as rust is a concern. In some embodiments, it may be preferable to use a weight bearing plastic, especially if weight and cost is a concern. One skilled in the art would appreciate that the strength of the materials used to construct the framework would need to be appropriate to adequately support the weight of the column(s) of reaction chambers. For example, if one were to construct a relatively small and inexpensive photobioreactor with only a few shelves, then a material such as plastic might be more appropriate. If on the other hand, one were to construct a large, durable photo-bioreactor for long-term industrial use, a material such as stainless steel or a powder coated metal might be more appropriate. The material may be flat, tubular or of some other appropriate shape.

The Modular Design of the Framework

The design of the framework is modular. As shown in FIG. 1 each framework unit will be designed with vertical support members 16, attached to longitudinal connecting members 18 and transverse connecting members 20.

The height of the vertical support member 16 can range from about 50 cm to about 8 m. In general, the height will range from about 2.4 m to about 3.6 m. In an embodiment, the height will range from 1.2 m to 4.8 m. The length of the photobioreactor can range from about 1.2 m to about 100 m long. In practice the length will be about 1.2 m to 50 m long. In another embodiment, the length varies between 0.6 m to 3.6 m long.

In embodiments where the microorganisms will be cultured within reaction chambers oriented in parallel, such that the culture media within one reaction chamber only enters and exits that chamber (without flowing through another chamber, positioned at a lower elevation) there will generally be a front end of the photo-bioreactor where the media and inoculum enter the reaction chambers and a back end where the culture media exits the reaction chamber upon harvesting. In some embodiments, the harvesting can be also accomplished at the end where the media, nutrients, gas, etc. enter the reaction chambers. In practice only 30% to 50% of the production is harvested at any time, leaving behind enough inoculum to grow for the next harvest. Harvesting can be achieved at either end of the photobioreactor, depending on which end better suits the overall design of the system and the environment in which it is positioned.

Each framework unit may have a rectangular footprint or a square footprint depending on the space available. Each framework unit supports one vertical rack of shelves, constituting one column of reaction chambers, when viewed from either the entry-end or the exit-end of the chambers.

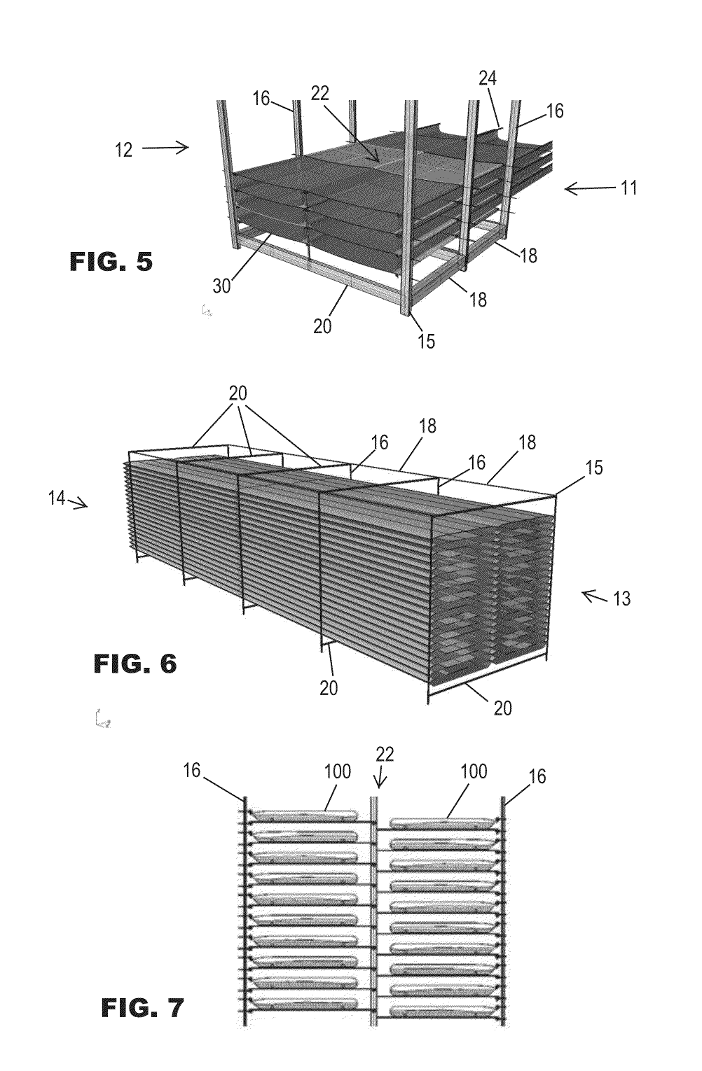

As illustrated in FIGS. 5, 6, 7 and 28, these framework units can be combined side-by-side to construct a photobioreactor with two vertical racks of shelves positioned side-by-side, with either a common central support, or with the inner supports connected in an appropriate manner. In certain embodiments as illustrated in FIG. 5, when the framework units are positioned side-by-side, the units share common shelves. Thus, each rack comprises a shelf 30 that extends from the outer edge of the first side 11 side of the first unit to the outer edge of the second side of the adjacent unit. When the shelves are constructed of a flexible material, the common central support 22, or connected inner supports, act as a central support system to prevent sagging which may otherwise occur when a flexible sheet is stretched and suspended across this distance. In some embodiments, the common central supports 22 between the two framework units are specifically configured to provide the central support system. In accordance with these embodiments, the two framework units share common vertical support members 16 on the inner side of the units. These vertical support members 16 are perforated to allow the common horizontal longitudinal connecting members 22 to pass therethrough. These common horizontal longitudinal connecting members 22 on the abutting inner sides of the units provide support to the flexible shelves 30 extended between first and second sides 11, 12 the photobioreactor rack framework 15.

It should be emphasized that shelves do not need to be waterproof but reaction chambers positioned above them must be waterproof. Therefore short-length shelves of framework units can be attached end-to-end to support long reaction chambers of any length.

As depicted in FIG. 1, wherein one embodiment is shown illustrating at least four modules combined end-to-end to form the framework of the photobioreactor, these framework units can be extended end-to-end, usually sharing common supports, to create a bioreactor that extends lengthwise as long as is practical. Given that the reaction chambers can be formed in any length that is practical, the reaction chambers extend as a singular chamber along the entire length of each shelf, with the entry-end and the exit-end located at opposite ends of the bioreactor.

In one embodiment of the photobioreactor 10, comprising two columns of shelves as depicted in FIG. 7, the shelves are arranged such that the shelves forming one column are either higher or lower than the shelves in the adjacent column. This embodiment might be desirable to be employed in situations wherein a longer effective reaction chamber length is to be achieved by flowing the microbial medium from one reaction chamber to another positioned adjacently and slightly lower than the originating reaction chamber.

In certain embodiments such as those depicted in FIGS. 2, 3 and 16, the framework further comprises an edge support 26 that engages a lateral edge 116 of the reaction chamber 100. The flexible reaction chamber 100 once filled with liquid tends to flatten and expand, and may thus extend beyond the edge of the shelf, with the result that this portion of the chamber is pulled below the shelf level by gravity. The edge support 26 prevents this from happening. An example of an edge support 26 in one embodiment is shown in cross-section in FIGS. 2, 3, and 16. In these embodiments, the lateral edge 116 of the reaction chamber 100 is folded around a suitable filler and inserted into a C-shape railing that is positioned on the vertical support member 16 of the framework at a suitable position above the shelf. Suitable fillers include, but are not limited to, various types of rope, such as foam rope, plastic rope, jute rope, cotton rope and the like.

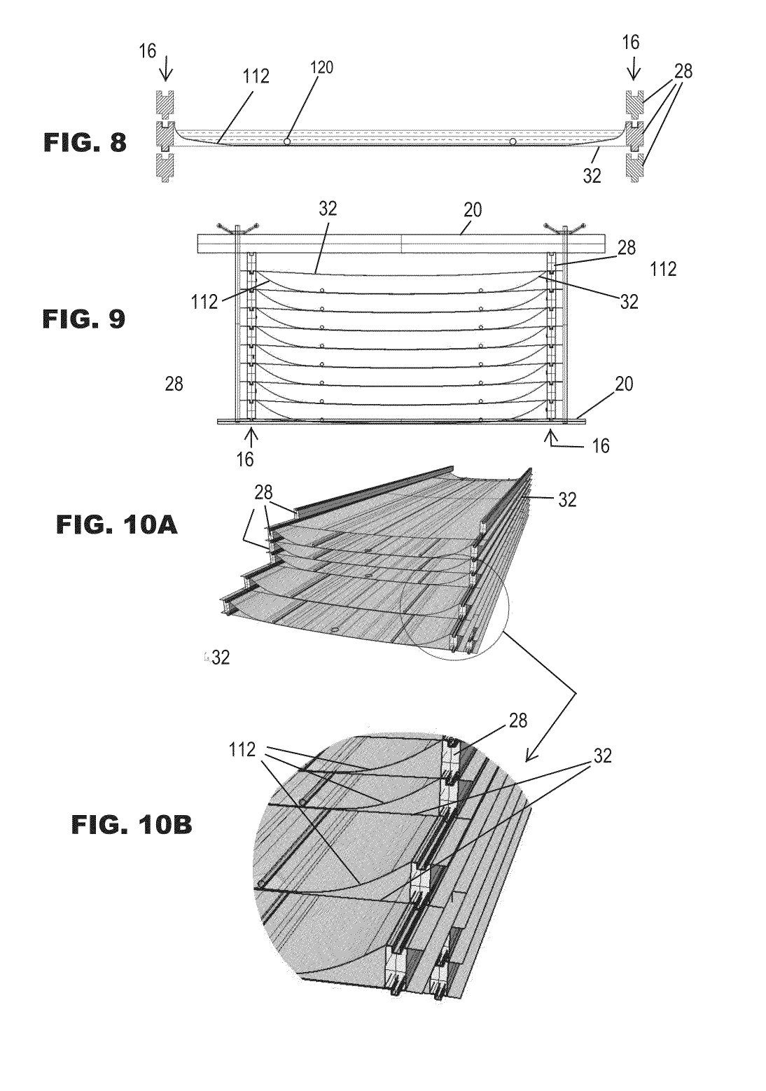

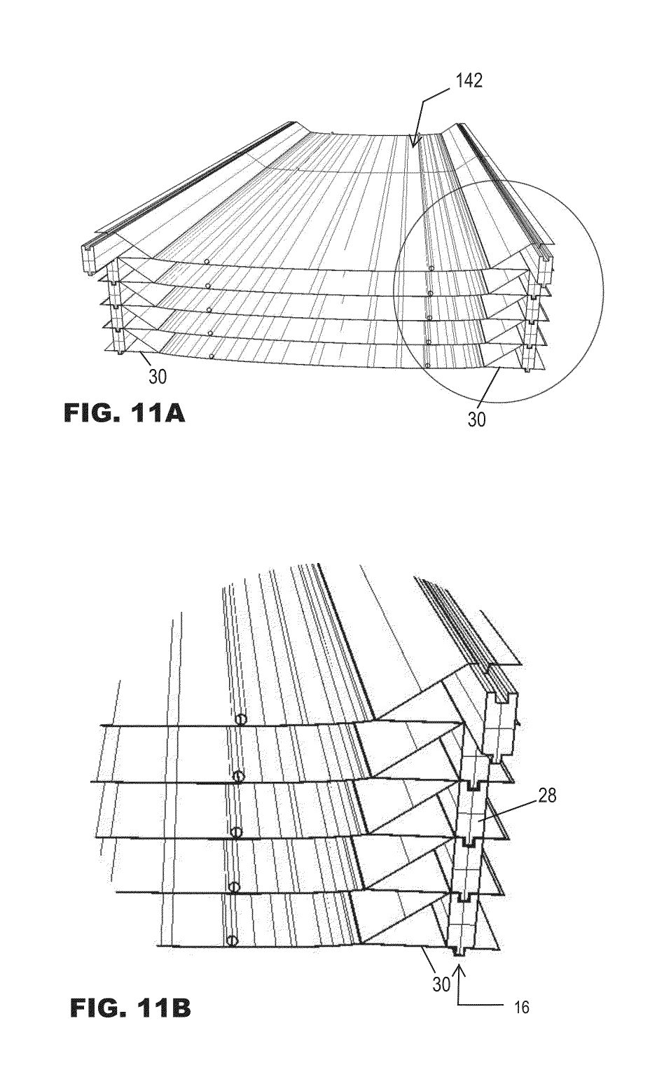

In some embodiments, the vertical support member may be provided as in modular format rather than as a single piece. An example of such an embodiment is shown in FIGS. 8, 9, 10A, 10B, 11A, and 11B. In this embodiment, the vertical support member 16 comprises a series of interlocking support members 28, with complementary protrusions and recesses, which once interlocked, provide strength and rigidity to the vertical support members 16. The sections also allow for insertion of a portion of the shelving material such that, once the sections are interlocked, the shelf is stretched tight between opposing vertical support members. Likewise, one edge of the reaction chamber may be inserted between sections of the vertical support member positioned above the sections holding the shelf, such that an edge support for the reaction chamber is provided.

The edge support also allows for creation of a space for vents to be inserted into the chamber to remove excess gases if needed (see below).

The Shelves

The shelves provide the horizontally oriented planar support for the reaction chambers. The vertical spacing of the shelves determines the density of the reaction chambers.

The Orientation of Shelves

The framework and the shelving can be constructed in a number of configurations in order to meet the specifications of the production facility.

In one embodiment, as illustrated in FIGS. 1A, 1B, 14, 17A and 17B, the framework structure can comprise a single vertical rack of shelves. The length of these shelves can be as long as is practical and may be supported by one or a plurality of framework units depending on the length of the shelves.

In one embodiment, as illustrated in FIGS. 5, 6, 7, 28, and 29, the framework structure can be constructed with shelves positioned side-by-side using a common vertical central support system to form two vertical racks, wherein the shelves are accessed from the external sides 11, 12.

In one embodiment depicted in FIG. 4, the shelf 30 is made of rigid material and comprises a cavity 32 that is longitudinally oriented along the edge of the vertical support member 16. When a reaction chamber is supported by this embodiment of a shelf the weight of the media will cause the bottom wall of the reaction chamber to drop into and fill this space, thereby creating a longitudinal channel within the reaction chamber containing the majority of the culture media. One or more sparger tubes positioned at the bottom of this channel will allow for increased gas dissolution within the culture media. To harvest the biomass within this embodiment, the channel portion of the reaction chamber is elevated causing a portion (around 30%) of the culture to overflow onto the horizontal section 36. Elevation of the channel portion of the reaction chamber is provided by filling a flat bag positioned under the reaction chamber with air or water such that the channel portion of the reaction chamber is elevated causing the culture media to move.

If water is used for elevating the channel portion of the reaction chamber, the same water used for elevating a portion of the reaction chamber positioned in the uppermost shelf is later directed to perform the same elevation function in a lower adjacent shelf. Opening and closing of valves is coordinated by a micro-computer controller.

In one embodiment, the structure can comprise two vertical racks of shelves, oriented side-by-side and separated by a space, such as a service passageway, which allows access from each internal side, if the bioreactor is contained within an outer protective cover such as a warehouse, a greenhouse or a shipping container.

A bioreactor may comprise a number of shelves ranging from at least two to about forty in number, for example, between 2 and about 35, between 2 and about 30, between 5 and about 30, between 10 and about 30, or between about 15 and about 30. In one embodiment, the number of shelves can range from 2 to about 40. In one embodiment, the number of shelves can range from about 19 to about 30.

The Shelf Materials

Depending on the requirements of the user, the shelves of the photobioreactor can be constructed with a variety of materials that meet the specifications of the installation.

In one embodiment, the shelves are made of transparent material. Shelf support made of stretched translucent plastic sheets, such as PETG sheets, benefit from light exposed both underneath of them and on top of them. The thickness of the sheets determines the amount of weight they can support. PETG sheets, for example, are commonly made of standard sizes of 4 feet by 8 feet (1.2 m by 2.4 m). These three factors (translucency, thickness and external sizes) may be varied.

In one embodiment, the shelves are made of a material that reflects light. In one embodiment, the shelves are made of some combination of transparent or light reflective material.

The shelves can be constructed out of rigid or semi-rigid materials. The materials that can be used to construct the shelves can be semi-rigid plastic sheets, fiber reinforced plastic, low density polyethylene, high-density polyethylene, nylon, hard acrylic, polyvinyl chloride, polycarbonate, composite plastic, ethylene vinyl acetate, fiber glass, woven fabrics, non-woven fabrics, stainless steel sheeting, powder coated steel and a combination thereof.

In one embodiment, the shelves are constructed from rigid materials such as fiber reinforced plastic, low density polyethylene, high-density polyethylene, nylon, hard acrylic, polyvinyl chloride, polycarbonate, composite plastic, ethylene vinyl acetate, fiber glass, stainless steel sheeting, powder coated steel and a combination thereof.

In one embodiment depicted in FIGS. 2, 3 and 16 shelves are comprised of transparent, semi-rigid plastic sheets secured to rigid metal extruded profiles such as, but not limited to square bars made of aluminum or stainless steel, by fastening means such as industrial staples. In some embodiments, these rigid metal profiles may also form the horizontal longitudinal connecting members 18 and are fastened to the vertical support members 16 of the framework by appropriate fastening means. Alternatively, the framework may comprise the rigid metal profiles in addition to the horizontal longitudinal connecting members 18, and the rigid metal profiles may be fastened to either the horizontal longitudinal connecting members 18 or the vertical support members 16 of the framework by appropriate fastening means.

The thickness of the shelves can vary as per the requirements of the system. Each shelf must be strong enough to bear the weight of the medium present in the portion of the reaction chamber that is positioned just above it while also providing sufficient tension to avoid excessive downward deflection. For example, in some embodiments, the shelf material and thickness is selected such that once installed as a shelf in the bioreactor, it allows less than 1 cm deflection along the length of the shelf.

In one embodiment, each plastic shelf is substantially about 0.010″ (0.254 mm) to about 0.080″ (2.032 mm) thick. In one embodiment the shelf is about 0.02″ (0.508 mm) to about 0.03″ (0.762 mm) thick. When made of stainless steel or of powder coated mild steel, sheets of at least gauge 24 (0.794 mm) can be used.

The Reaction Chamber

The reaction chamber 100 is constructed from a pliable durable translucent material that allows for the transmission of light and the ability to gusset the material to construct tubes 122, 124 running along the wall of the reaction chamber. Reaction chambers may be formed, for example, by cutting desired lengths from rolls of material that has been preformed into reaction chambers containing delivery tubes. Reaction chambers may be, for example, around 3 feet wide flat tube (90 cm) with length varying from 4 feet (1.2 m) to 262 feet (80 m). In certain embodiments, the width of the reaction chambers can range in size varying from about 0.5 m to about 4 m. In general, reaction chambers will be sized from about 1.2 m to about 2.4 m in width. In other embodiments, reaction chambers may be sized from about 0.60 m to about 1.8 m in width and 50 m long. To prevent medium loss from both ends, tube ends are elevated slightly above water level.

In one embodiment, the reaction chamber is sealed at one end. Inoculation and harvesting are processed from the same end using the same principle as described earlier.

As shown in FIGS. 2, 3, and 16 in certain embodiments, at least one elongate border portion of the chamber 100 is elevated above fluid level and secured to an edge support 26. This configuration can assist in removing oxygen from the reaction chamber as it creates space for vents to be inserted into the reaction chamber 100 to vent excess gases.

Fluid level and flow from one or multiple reaction chambers 100 located on a higher shelf to sleeves located on adjacent lower shelves as illustrated in FIG. 7 may be controlled via height-adjustable fluid exit means known in the art, such as height-adjustable valves. These can assist in establishing the desired level of fluid in each reaction chamber before extra fluid overflows to another destination. The valves may be positioned, for example, at one or both ends of each reaction chambers.

In certain embodiments, the reaction chambers comprise one or more overflow valves to maintain the fluid level within the chamber within a predetermined level. In some embodiments, the overflow valve is positioned in the bottom of the reaction chamber and extends through the base wall of the chamber and through the shelf below. The walls of the valve sealingly engage the wall of the chamber to prevent fluid escaping and an open end of the valve extends above the predetermined fluid level within the reaction chamber. An increase in fluid level will result in the fluid level rising above the open end of the valve and fluid being removed therethrough. The valve may be operatively associated with a disposal means, such as a tube or other conduit that conducts the overflow fluid to another location.

The Material

The reaction chambers are constructed from a translucent pliable material, enabling the material to be gusseted to form delivery tubes that can be used as sparger tubes and /or tubes for the delivery of nutrients. The materials that can be used to construct the reaction chambers can be at least one of the materials selected from the group consisting of: fiber reinforced plastic, low density polyethylene, high-density polyethylene, nylon, hard acrylic, polyvinyl chloride, polycarbonate, composite plastic, ethylene vinyl acetate, fiber glass, woven fabrics, non-woven fabrics and a combination thereof.

The thickness of the wall of the reaction chambers 100 is about 2 Mil (50.8 micron) to about 12 Mil (304.8 micron) thick. In certain embodiments, the thickness of the wall of the reaction chambers 100 is about 4 Mil (101.6 micron) to about 8 Mil (203.2 micron) thick.

The Delivery Tubes

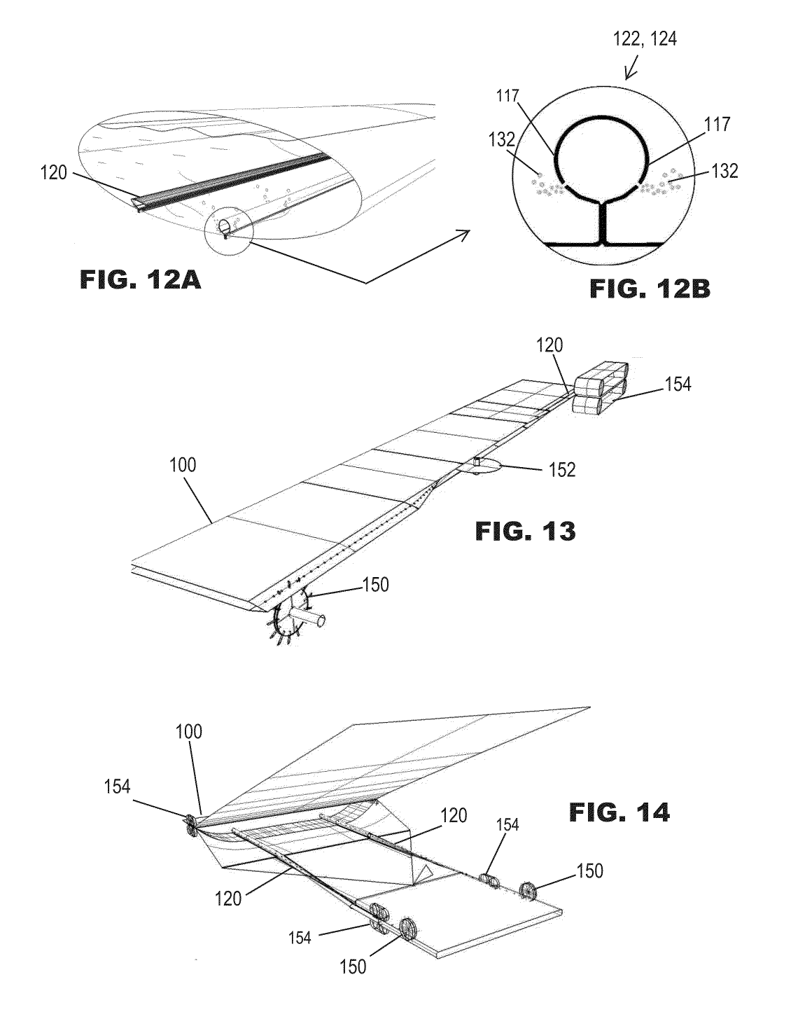

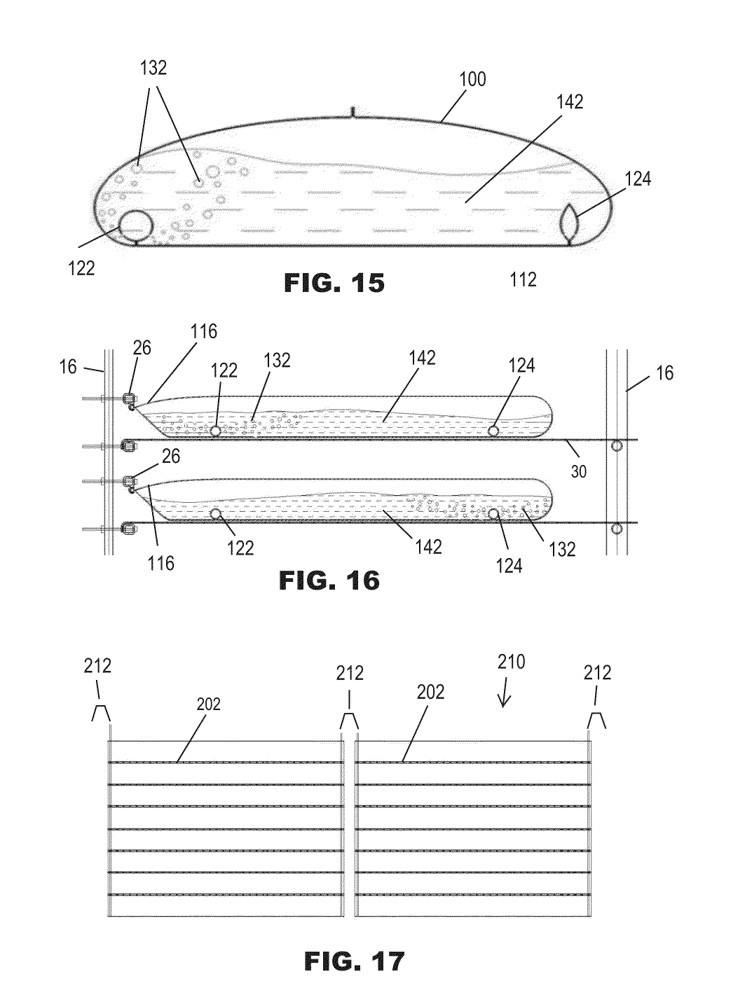

Longitudinal tubes can be constructed from the material that will constitute the wall of the reaction chamber in a manner that the tubes can be used to deliver gas and other nutrients to the biomass growing within. As illustrated in FIGS. 12A and 15, one embodiment of the reaction chamber 100 can be designed to have one delivery tube 120 designed for the delivery of nutrients or other substances to the culture medium, or for the delivery of gas in sparger tubes 122, 124.

In one embodiment of the invention, the number of holes per surface area is the same along the full length of delivery tube. In another embodiment, the number is intermittently variable along the tube. In yet another embodiment, two, three or more holes are punctured concurrently along a perforation line. In one embodiment, the radial position of holes is the same, while in another embodiment the position is varied along the length of delivery tube. In one embodiment, the diameter of delivery tube is varied along the length of the tube according to a pre-determined pattern.

In certain embodiments, holes 117 are perforated or punctured by a single puncturing action that concurrently punctures the two adjacent walls present in each fold. The diameter of the resulting tube can vary as required for the design of the system and the fluid or gas that will be delivered through the tube.

FIGS. 13 and 14 illustrate how a perforator such as, but not limited to, a needled wheel, a laser cutter, a waterjet cutter, a pneumatic punch or any other perforator means of the like may perforate from any one side of a folded, flexible, puncturable plastic sheet two holes 117 in a single step.

FIG. 13 shows one method of creating a delivery tube 120 in a thin film plastic wherein the perforated portion of the wall of a reaction chamber is tucked into the same material to create a gusset using the gusseting roller. This method is a one-sheet method. After proceeding with a blown film extrusion process, one wall of the reaction chamber is drawn over perforating equipment 150, such as a punch, a needle wheel or a laser cutter. The perforated portion is then drawn into gusseting wheel 152 that tucks in the perforated portion into the reaction chamber before a sealing machine 154 bonds the newly formed edge. Sealing may be performed using ultrasonic, heat or radiowave welding. The same method applies for shaping two delivery tubes in the same reaction chamber. To achieve this, additional equipment is positioned in a mirror position than for building one delivery tube. To shape three or more delivery tubes in a same reaction chamber, the method requires to re-fold the wall of the reaction chamber in a manner where new fold edges are created and re-apply the same tube-shaping method. To seal external edges of the gusseted portion, a band sealer or any heat sealer of the like may be used.

FIG. 13 shows one method of creating a delivery tube 120 in a two-sheet method. The method consists in forming one or two delivery tubes along the lateral sides of an elongate sheet that originally may have been a closed sleeve or a sheet folded on both sides as shown in FIG. 13. The longitudinal edges of a reaction chamber are each perforated by perforating equipment 150. The bottom wall of the reaction chamber is then cut open by a knife and edges of the chamber are drawn upward to meet an upper sheet. In a final step, opposite edges of both sheets are sealed together to form a new chamber that encloses the two delivery tubes.

The Sparger Tubes

In one embodiment of the invention the introduction of gas into a liquid is accomplished via a delivery tube, which can be configured as a sparger tube. Sparger tubes in certain embodiments as depicted in FIGS. 2, 3, 12A, 12B, 15 and 16 are designed in a manner that sparges gases from two oppositely-oriented holes 117 such that gases will exit the delivery tube in opposite directions, perpendicular to tube 122, 124 and slightly downwards. In this embodiment the holes 117 that emit the gas are located close to the tube's sealing line 114 in contact with the bottom wall 112 of the reaction chamber 100.

In one embodiment of the invention, the bottom wall 112 of the reaction chamber 100 incorporates two sparger tubes 122 and 124 for dispensing gases along the full length of the reaction chamber 100.

The Fluid Agitation System

In certain embodiments, the bioreactor comprises a sparger system that generates aeration and mechanical agitation in a single process. This is achieved by alternately pressurizing each of the two or more sparger tubes provided with their holes positioned facing the bottom wall of the reaction chamber. FIG. 15 shows a reaction chamber having two sparger tubes 122 and 124 in operation. As shown, the shape and size of a fully blown sparger tube 122 in FIG. 15 varies from the shape of a more contracted sparger tube 124 shown at the right side of reaction chamber 100. Therefore, in addition to the agitation created by the sparging and bubbling effect, this shape variation of the sparger tube 122 increases the amount of mechanical agitation and mixing provided by the sparger tube 122, 124.

An electronic switching system turns on and off air pressure between the two sparger tubes creating pressure variations in sparger tubes 122, 124 resulting in a transverse harmonic agitation along the full length of reaction chamber 100 creating waves that mix intimately gases with the algal medium. Moreover, as illustrated in FIG. 16, the physical vibration of air exiting the sparger tubes 122, 124 is used as a source of agitation, adding to the agitation created by bursting bubbles 132 that exit from same tubes 122, 124. To increase vibration, a pressure pulsation similar to “water-hammer” in liquids is created in sparger tubes 122, 124.

Vibrations may be also be generated using a venturi effect caused by releasing a pressurized but un-even air flow via small orifices positioned along the two sides of the thin air sparger tubes 122, 124 positioned under algal medium.

Volume and pressure fluctuation of gases generated in sparger tubes 122, 124 is created by adding to an air or carbon dioxide gas delivery system a means such as, but not limited to, modified diaphragm, a floating tongue, an unbalanced or balanced rotor, an unbalanced or balanced propeller, an electrically-driven modulator or a combination thereof.

The Nutrient Delivery System

Nutrients may be delivered to the reaction chamber by a range of small external delivery tubes or by internal delivery tubes built into the reaction chambers. FIG. 12A illustrates an embodiment wherein a delivery tube 120 is used to deliver nutrients to the reaction chamber 100. The amount of nutrient that is delivered is controlled by a computerized controller that operates a peristaltic pump. To determine how much nutrient is required, a number of sensors log continuously the amount of oxygen released by the culture under what temperature, pH and carbon dioxide levels (in the form of water dissolved carbon) all based on a known amount of delivered nutrients. This knowledge verifies the amount of nitrogen and carbon that has been delivered to the culture and is matched against the amount of nitrogen and carbon present in the harvested biomass. This collection of knowledge is translated into a single reading of the amount of oxygen released daily by the biomass, which in turn will determine the amount of nutrient the pump will deliver.

The Illumination System

The photobioreactor 10 includes a source of artificial light such as LED lights, which may be in the form of LED tapes 202, LED bars, LED lamps, LED mats 210 or LED tubes, or the like. The lighting system may be external to the reaction chambers or it may be an internal lighting system positioned within the chamber(s). For embodiments where the illumination system is exterior to the reaction chambers, the light source(s) may be positioned above or at the side of the reaction chamber 100 or some combination thereof. In some embodiments, the source of light may also include solar light in combination with artificial light. In one embodiment, the LED lights are contained within translucent plastic tubes and placed within the reaction chambers, oriented along the length of the chamber.

In one embodiment shown in FIG. 17, the light source comprises multiple parallel rows of LED tapes 202 that are positioned over a mat 210. They are provided with select wavelengths that enhance algae growth. LED bulbs are either embedded, sandwiched and laminated between two transparent films to form collectively a wide, flexible, modular transparent mat 210 that may be electrically connected via connectors 212 among themselves and powered by an electrical source.

While the above description specifically mentions LED lights as an artificial light source, one skilled in the art will appreciate that other light sources capable of providing light at photosynthetically active radiation (PAR) wavelengths of about 400 nm to 700 nm are also suitable.

In one embodiment the reaction chambers are exposed to solar light 1000. To prevent unwanted light waves such as ultra-violet and infra-red (UV and IR) lights to negatively affect algae growth, the transparent or translucent material that comprises the reaction chamber 100 may be adapted to allow only photosynthetically active radiation (PAR) wavelengths of about 400 nm to 700 nm to reach an algal medium contained in the reaction chamber 100.

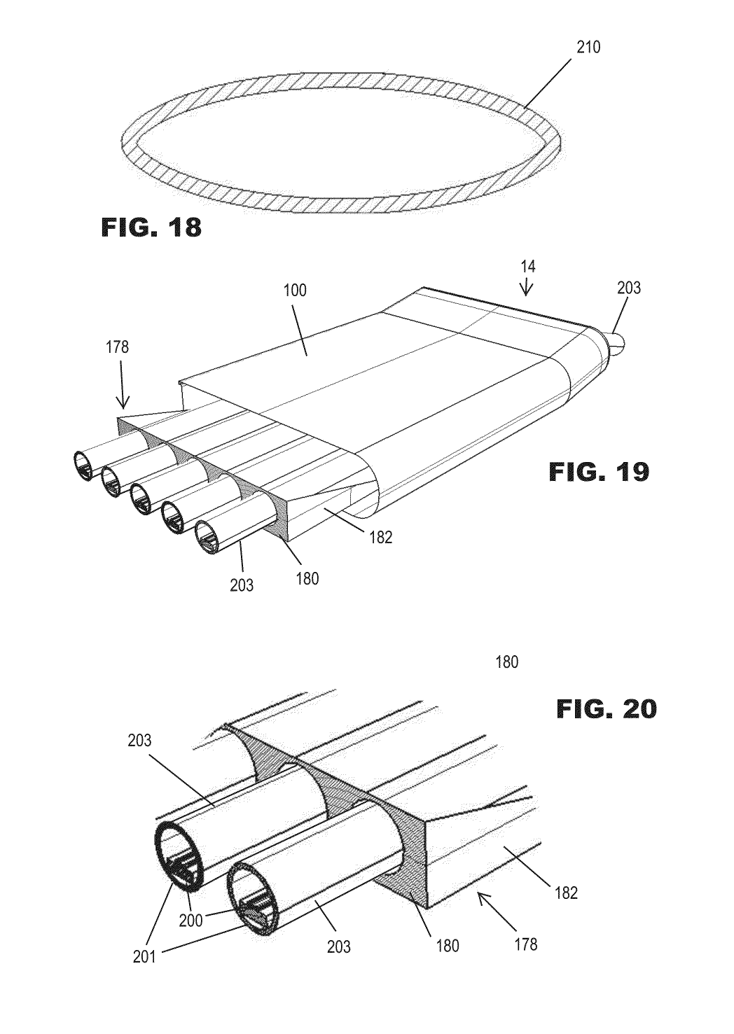

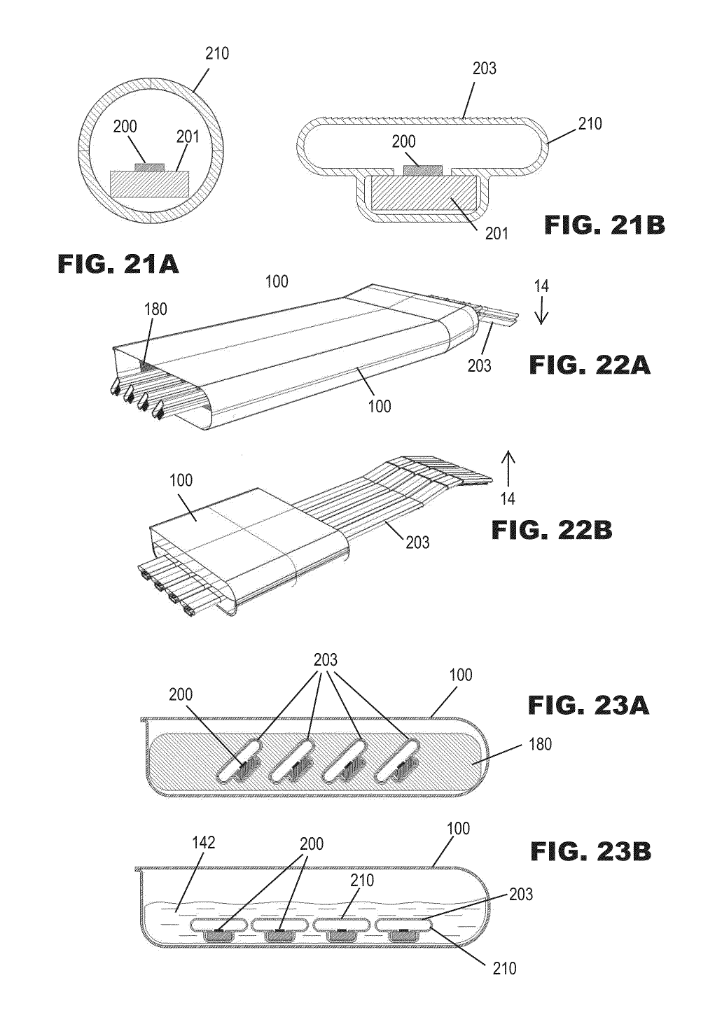

Embodiments of the photobioreactor comprising light tubes 203, comprising an LED 200 operatively mounted on a longitudinal LED support 201 placed within a tubular cover 210, positioned within the reaction chambers are illustrated in FIGS. 19, 20, 22A, 22B. FIG. 18 shows the shape of an oval tubular cover 210 within which LEDs 200 positioned on a LED support 201 can be inserted, as shown in FIGS. 21A and 21B to generate light tubes 205. FIG. 21A illustrates a circular light tube 203 and FIG. 21B shows a flat light tube 203, wherein the tubular cover 230 has been conformed to the shape of the LED 200 on the LED support 201.

FIG. 19 illustrates an embodiment wherein circular light tubes 203 are positioned within the reaction chamber 100. FIG. 19 also shows the light tubes 203 are engaged within a biomass collector plate 180, held in a vertical position by biomass collector arms 182.

The biomass is harvested by pulling the biomass collector arms 182 along the light tubes 203 such that the biomass collector plate forces the biomass to move with the biomass collector 178 towards the second end 14 of the photobioreactor. The holes within the biomass collector plate encasing the light tubes are sized such that they closely engage with the outer surface of the light tubes. As the biomass collector plate 180 moves along the longitudinal exterior surface of the light tubes 203, the biomass growing on the surface is scraped from the surface and drawn towards the second end 14 of the reaction chamber 100. The light tubes are angled upwards near the second end 14 of the reaction chamber 100 such that the majority of the water in the culture media remains within the reaction chamber 100 and is partially dewatered.

FIGS. 22A, 22B, 23A and 23B demonstrate an embodiment that is similar to the one in FIG. 19 except that the flat light tubes 203 are illustrated. In this embodiment the holes within the biomass collector plate 180 are angled or vertical in order to enable the biomass collector plate to clean the entire exterior surface of the flat light tubes 203. FIG. 23B shows how the flat light tubes are positioned at the bottom of the reaction chamber.

Means for Delivering and Distributing Inoculum

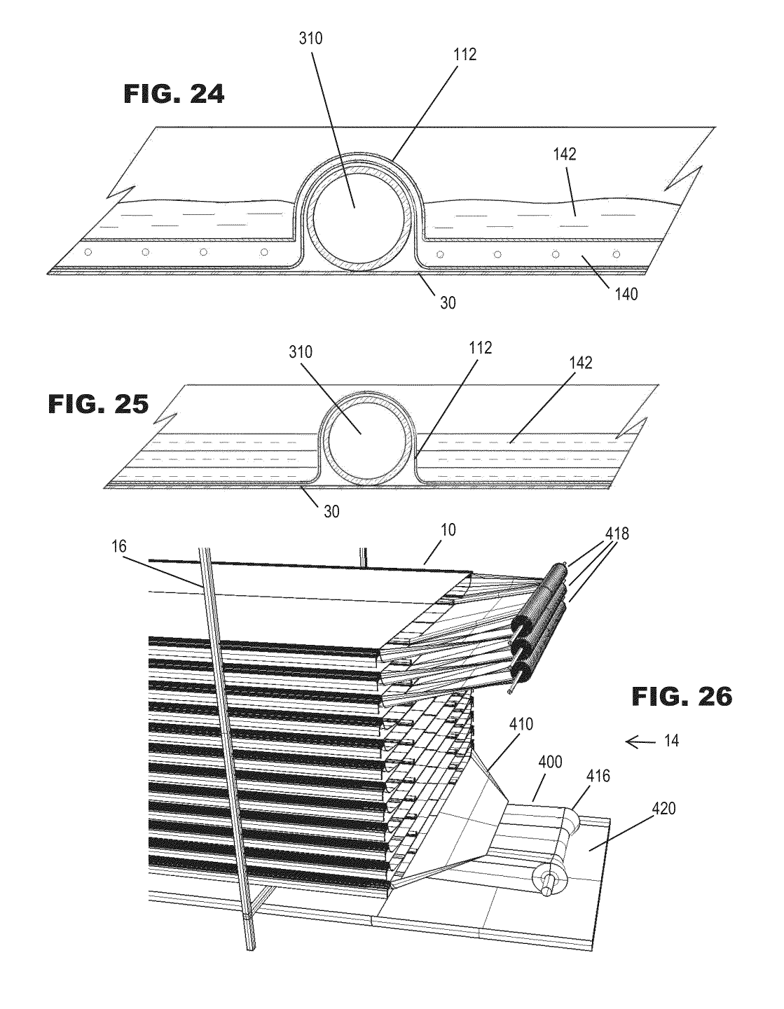

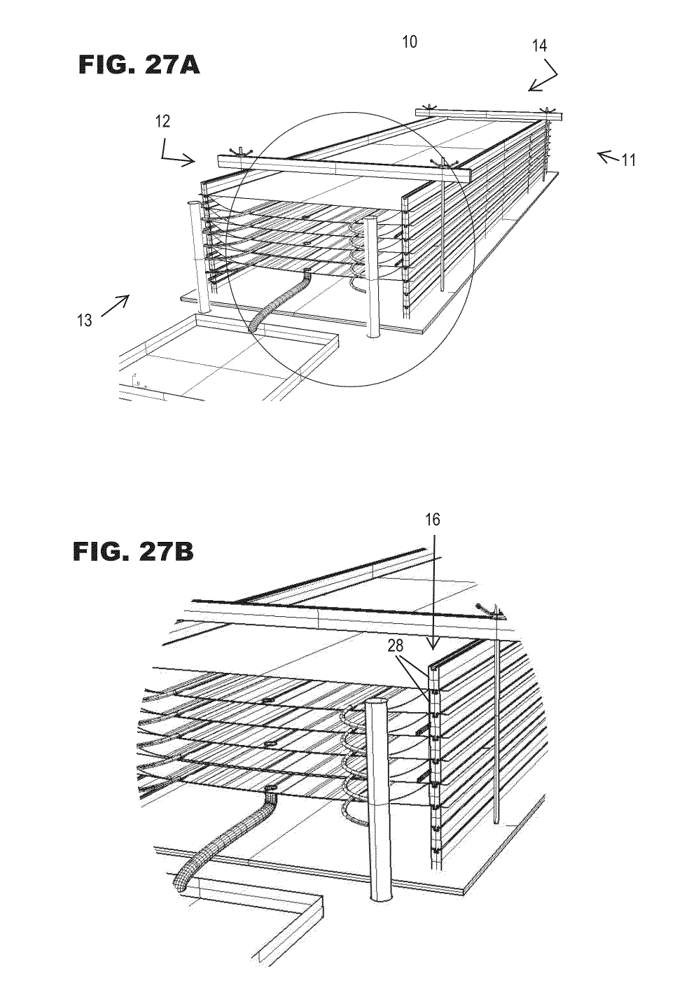

To expand inoculum gradually, controllably and safely without being exposed to contamination, especially during transfer of the culture medium to larger sized containers, has been a challenge among algae farmers. As one means of addressing this challenge, the photobioreactor may in some embodiments comprise a movable divider 310 as illustrated in FIGS. 24 and 25, which is positioned underneath the bottom reaction chamber wall 112 that enables a full volume of medium 142 contained in each of the reaction chambers 100 to be divided, then isolated and then expanded in a controlled manner. FIG. 24 shows an embodiment wherein the reaction chamber has a thermal regulator 140 positioned between the bottom reaction chamber wall 112 and the shelf 30. Control of expansion is achieved by rolling or sliding one or multiple movable dividers 310 that are positioned under the bottom reaction chamber wall 112 of the flexible reaction chamber 100. The dividers 310, which elevate and isolate a portion of the reaction chamber 100 include, but are not limited to, movable rollers 310, liftable bars, roller-over-bars, stretchable bungees, ropes, cables, raisable panels, slidable self-standing dividers and a combination thereof.

Parallel Processing of Reaction Chambers

Having multiple reaction chambers so densely located next to each other enables one to subject algae to collaborative processes including extreme environmental conditions and shocks that stimulate algae reaction. Such environmental treatment may include subjecting the algae to high or low electromagnetic fields, high or low flashes of light, flashes of heat, exposure to sound waves, and a combination thereof. As an example, a plurality of upper levels of reaction chambers may be engaged in culture of an algae species receiving air, CO₂, agitation and nutrients, whereas one or more lower levels of algal medium may be cut off from air and nutrients to undergo a starvation process forcing them to transform their biomass into oil, and finally one or more of the lowest levels may be used as transfer containers to maintain continuity of the process.

To engage different individual chambers or groups of reaction chambers to perform different tasks or processes in a same photobioreactor, valves may be opened or closed manually or automatically enabling fluid flow in a reaction chamber in a vertical downward direction (for example to maintain fluid at a predetermined level in the chamber), in a horizontal sideways direction from left to right or vice-versa (for example, between adjacent reaction chambers), or follow a pattern programmable by a controller means (not shown).

In the disclosed photobioreactor, biofilm or deposits from sedimentation in a reaction chamber may be removed by displacing manually, automatically or by pressure differential means a movable cleaning pig (not shown) in the reaction chamber. To achieve this, a mop-shape cleaning pig attached to two ropes, one on each side of the cleaning pig, may be pulled.

The Optional Dewatering System

In certain embodiments, the bioreactor may optionally comprise a dewatering system, which can be used to significantly reduce the water content of the biomass. In one embodiment of the photobioreactor shown in FIG. 26, a biomass dewatering system 400 is provided. The biomass dewatering system 400 comprises an elongate motorized conveyor belt located in a trough 420 that is positioned at the lowest level of photobioreactor 10; the conveyor belt 416 of the also functions as a filter adapted to receive and partially dewater biomass. A funnel 410 collects the biomass exiting the reaction chambers and directs it towards the conveyor belt 416. In one embodiment there are multiple funnels 410 positioned at different elevations in order to collect the biomass as it exits each reaction chamber 100 or multiples of reaction chambers 100. The conveyor belt 416 is exposed to a heating zone that dries and transforms the thin layer of biomass into a peelable crust that can be collected by gravity.

In one embodiment, for example, the algal culture in any of the reaction chambers may be dewatered by directing flow of the culture towards a filtration chamber positioned at a lower level wherein a natural filtration by gravity may take place. Thus reducing substantially the amount of energy associated with dewatering.

The Thermal Control System

In certain embodiment, the bioreactor may include a thermal control system to maintain the temperature of the culture within the reaction chamber(s) within a predetermined range. In one embodiment of invention as illustrated in FIG. 3, thermal control of the reaction chamber 100 is provided by circulating a thermal fluid 142 through a thermal regulator 140 that will heat or cool the reaction chamber. The thermal regulator 140 is created by positioning an elongate bag just below the shelf 30 supporting a reaction chamber, the thermal regulator support 32 being constructed from a flexible material stretched underneath the shelf 30 and secured by the same attachments as the shelf 30 supporting the reaction chamber 100. In embodiments with flexible shelves, the shelf 30 and the thermal regulator support 32 can be made by doubling the flexible material of the reaction chamber and securing them to the sides of framework using the same attachment means, with the upper shelf 30 being tightly stretched and the lower support 32 being loosely stretched.

The upper tightly stretched panel creating the shelf 30 is sized to be of slightly shorter length that of the lower loosely stretched panel creating the thermal regulator support 32. In such an arrangement, the lower loosely stretched panels form an elongate surface over which may be extended a long flexible or semi-rigid shallow thermal fluid chamber 160. Circulating a slightly pressurized thermal fluid 142 (i.e., hot or cold fluid) in said thermal fluid chamber 160 causes the chamber to press upwardly against the bottom of the upper tightly stretched panel forming the shelf 30 and exchange its heat or cold with reaction chamber 100. The depth of the thermal fluid chamber 160 may vary between about 10 mm to about 50 mm at its lowest point.

In one embodiment of the invention, cooling of the reaction chamber is achieved by evaporating dew very slowly seeping out from the upper reaction chamber wall 13. To optimize cooling by evaporation, the reaction chamber 100 is having, preferably an upper reaction chamber wall 113, a bottom reaction chamber wall 112, or both walls 112, 114 made of a transparent waterproof material that is breathable to enable very slow evaporation of condensates under a warm climate.

The Optional Fluid Collector

In certain embodiments, the bioreactor may optionally comprise a fluid collector to collect spills, overflow, leaks and the like. In some embodiments, the fluid collector channels and collects water from spills or leakage from the reaction chambers, and comprises a waterproof sheet loosely stretched between the opposing sides of the framework, positioned at the lowest level of the photobioreactor. The waterproof sheet is further provided with a drainage means (such as a funnel collector) connected to hoses that carry water spills away.

Optional Processing Systems

In certain embodiments, the photobioreactor provides a controlled environment in which multiple parallel or serial processes may occur within a reaction chamber of the photobioreactor itself or in association with equipment and accessories that are in air or fluid communication with reaction chamber, or are introduced in said reaction chamber. As an example, in some embodiments, the reaction chamber may be configured to provide internal layers that function as filtering membranes having their own sparger tubes with holes along their full length. When dealing with fluids of different densities, internal layers may transfer or filter fluids via osmosis or reverse osmosis.

In another example, introducing venturi jets into the algal medium creates micro bubbles that separate solids from liquids and lift agglomerated cells to the fluid surface making harvesting of microalgae easy as a simple skimming process. Other processing steps within the bioreactor reaction chamber may include electro flocculation, bioflocculation, biofloatation, fermentation, lysing, hydrogenation, localized heat treatment, localized light flash treatment, localized high or low magnetic field treatment; some of said processes causing stresses that may increase biomass productivity or influence it; yet another example include oil extraction by fracturing cells walls with cavitational micro-bubbles, and a combination of multiple processes mentioned before.

The Biomass Harvesting System

In certain embodiments, the bioreactor is configured to provide a simple means of biomass harvesting. As an example, a dewatered reaction chamber portion may be gradually pulled out of the bioreactor shelves, sealed and then separated into small packages. This one-step culturing and packaging guarantees avoidance of contact with air or other external sources of contamination. In a further step, the sealed packages may be safely transported, frozen or directly sold to consumers or to buyers.

The BioMass Monitoring System

The compactness provided by the present multilevel bioreactor improves monitoring and control of factors that influence generally the operation of a bioreactor. Such factors include temperature, light, pH, agitation, gas flow and liquid flow and physical factors of the like.

Reaction Chamber Replacement System

In one embodiment of the invention illustrated in FIG. 26, replacement of disposable or re-usable reaction chambers 100 is made easy by providing a stand (not shown) holding multiple rolls of reaction chambers 418. The stand is located at one end of photobioreactor 10 for holding a supply of rolls of reaction chambers 418. Pulling out, from one end of the photobioreactor 10, an old reaction chamber causes a supply of fresh reaction chambers to be dispensed from a corresponding roll of reaction chambers 418 located at the opposite end of the photobioreactor. Thus an empty or partially dewatered reaction chamber may be easily and readily (after dewatering) replaced by a fresh un-contaminated reaction chamber. Replacement of an older reaction chamber may take place because of damage, leakage, contamination, wear and tear, loss of clarity or as part of a processing step wherein biomass contained in the reaction chamber may be collected or packaged and further processed.

The Optional Outer Protective Cover

In some situations, it may be desirable to include an outer protective cover to the photobioreactor. Such a cover may be as simple as a tarp securely attached over the outer frame, or as secure as a steel container, such as a shipping container.

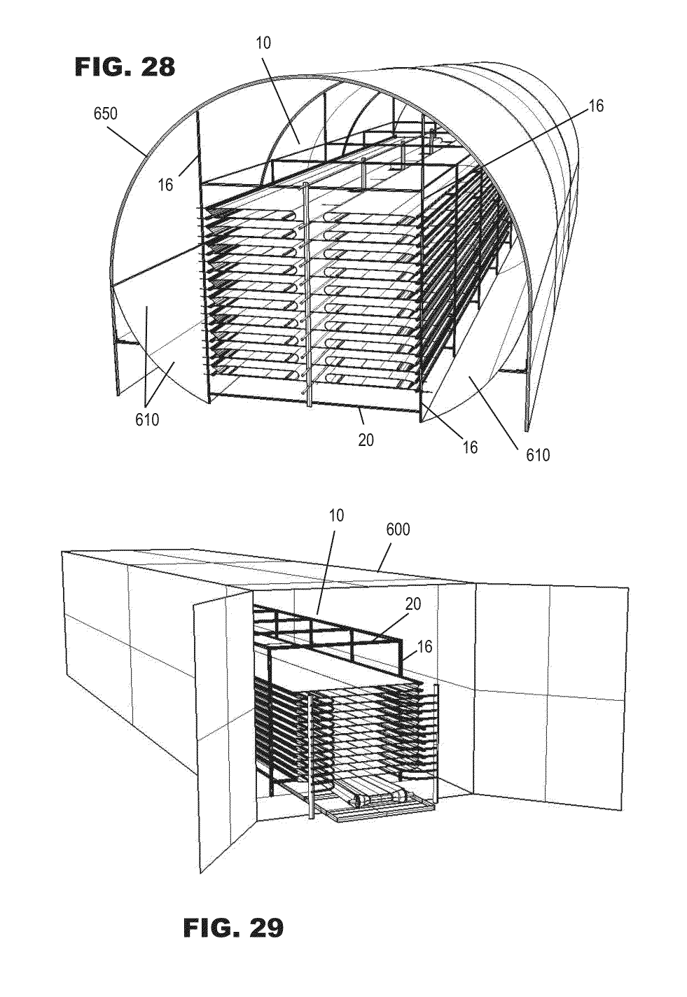

Some embodiments of photobioreactor 10 are adapted to operate indoors, inside a warehouse, a shipping container 600 (see FIG. 28) or inside any other closed structure or building.

In one embodiment of the invention, the multilevel bioreactor is surrounded by a circular-shape greenhouse 650 in which the lower portion of the cover close to the ground takes on a parabolic shape covered by a reflective material 610.

In one embodiment as illustrated in FIG. 28, the photobioreactor is configured to operate outdoors, protected from weather conditions under structures such a greenhouse 650 or an inflatable structure. In one embodiment, the photobioreactor is located at the center of the tunnel-shape greenhouse 650 with the cover being adapted to provide optimum photosynthetically active radiation (PAR) within wavelengths ranging about 400 nm to 700 nm with about 95% light diffusion. The lower portion of the greenhouse 650 is provided with a reflective portion 610 having a parabola-shape configuration. The reflective material comprising the reflective portion 610 may be flexible or semi-rigid and is adapted to reflect incoming light towards the reaction chambers 100.

In one embodiment, the outer protective cover is a geodesic building.

In one embodiment of the photobioreactor 10 is encased and installed in a steel structure such as a shipping container 600. Grouping multiple shipping containers together can quickly scale-up the production capability of an algae production facility.

1. A photobioreactor for culturing phototrophic microorganisms comprising:
a plurality of reaction chambers composed of a translucent, pliable, water-impermeable material, each of the reaction chambers being an elongate sleeve capable of holding a culture medium, and
a modular support structure comprising a framework defining first and second sides and a first and second end, and configured to support a plurality of horizontally oriented, vertically spaced shelves, each of the shelves extending from the first to the second side of the framework and having disposed thereon one of the plurality of reaction chambers.

2. The photobioreactor according to claim 1, wherein each of the reaction chambers comprises one or more delivery tubes for delivering nutrients and gas to the culture medium.

3. The photobioreactor according to claim 2, wherein the one or more delivery tubes are provided by one or more internally formed perforated gussets in a bottom wall of the chamber.

4. The photobioreactor according to claim 2, wherein each of the reaction chambers comprises two delivery tubes which are provided by two internally formed perforated gussets in a bottom wall of the chamber.

5. The photobioreactor according to claim 4, wherein one of the delivery tubes is operatively associated with a gas supply system and the other delivery tube is operatively associated with a nutrient supply system.

6. The photobioreactor according to claim 4, wherein both delivery tubes are operatively associated with a gas supply system.

7. The photobioreactor according to any one of claims 1 to 6 further comprising one or more nutrient supply systems operatively associated with each of the plurality of reaction chambers.

8. The photobioreactor according to any one of claims 1 to 7 further comprising one or more gas supply systems operatively associated with each of the plurality of reaction chambers.

9. The photobioreactor according to any one of claims 1 to 8 further comprising an illumination system operatively associated with one or more of the plurality of reaction chambers to provide light thereto.

10. The photobioreactor according to claim 9, wherein the illumination system comprises light emitting diodes.

11. The photobioreactor according to claim 10, wherein the light emitting diodes are located within each reaction chamber.

12. The photobioreactor according to claim 11, wherein the light emitting diodes are comprised by translucent tubes disposed along the base of each reaction chamber.

13. The photobioreactor according to claim 12 further comprising a biomass collection device operatively associated with the translucent tubes for removing biomass therefrom.

14. The photobioreactor according to any one of claims 1 to 13, wherein the plurality of shelves are composed of a rigid material.

15. The photobioreactor according to any one of claims 1 to 13, wherein the plurality of shelves are composed of soft plastic sheets.

16. The photobioreactor according to claim 15, wherein each of the shelves form a top wall of the reaction chamber disposed on the shelf below.

17. The photobioreactor according to any one of claims 1 to 15, wherein each of the plurality of reaction chambers is an elongate cylindrical plastic film sleeve.

18. The photobioreactor according to any one of claims 1 to 17, wherein the plurality of shelves are transparent or translucent.

19. The photobioreactor according to claim 17, wherein the framework further comprises a plurality of edge supports extending from the first to the second end of the framework, each of the edge supports configured to hold an edge of a reaction chamber and positioned relative to a shelf such that when holding an edge of the reaction chamber disposed on the shelf, the edge of the reaction chamber is in an elevated position.

20. The photobioreactor according to claim 19, wherein the edge support is a C-shaped rail sized to hold an edge of the reaction chamber together with a filler.

21. The photobioreactor according to any one of claims 1 to 18, wherein the framework comprises a series of interlocking support members on each side of the framework, each of the interlocking support members extending from the first to the second end of the framework, and positioned to allow an edge of a shelf and/or an edge of a reaction chamber to be inserted between adjacent interlocking support members and gripped thereby when the adjacent interlocking support members are in an interlocked position.

22. The photobioreactor according to any one of claims 1 to 21, further comprising a harvesting apparatus operatively associated with the plurality of reaction chambers for removing biomass therefrom.

23. The photobioreactor according to any one of claims 1 to 22, further comprising a thermal regulator operatively associated with each of the reaction chambers for regulating the temperature of the culture within the reaction chambers.

24. The photobioreactor according to claim 23, wherein the thermal regulator comprises a plurality of thermal regulation chambers, each of the thermal regulation chambers disposed below and in thermal contact with a bottom wall of a reaction chamber and configured to receive a thermal fluid.

25. The photobioreactor according to any one of claims 1 to 24, wherein the modular support structure comprises two framework units disposed side-by-side.

26. The photobioreactor according to claim 25, wherein the two framework units share a common central support.

27. The photobioreactor according to claim 26, wherein the common central support comprises a plurality of horizontally disposed support members that extend from the first to the second end of each framework unit and each of the plurality of shelves extends over one of the horizontally disposed support members and across both framework units.

28. Use of the photobioreactor according to any one of claims 1 to 27 for culturing phototrophic microorganisms.

29. A method of culturing phototrophic microorganisms comprising the steps of:
introducing phototrophic organisms into one or more reaction chambers of the photobioreactor of any one of claims 1 to 27 to provide a culture of phototrophic microorganisms, and
supplying gas and nutrients to the one or more reaction chambers.

30. The method according to claim 29, wherein the reaction chambers comprise one or more delivery tubes that are provided by one or more internally formed perforated gussets in a bottom wall of each chamber, and the gas is supplied to the reaction chambers through at least one of the one or more internally formed perforated gussets.

31. A reaction chamber for culturing phototrophic microorganisms, the reaction chamber composed of a transparent, pliable, water-impermeable material formed into an elongate cylindrical plastic film tube and comprising one or more internally formed perforated gussets in a bottom wall of the chamber.

32. A method of culturing phototrophic microorganisms comprising the steps of:
introducing phototrophic organisms into the reaction chamber of claim 31 to provide a culture of phototrophic microorganisms, and
supplying gas and nutrients to the reaction chamber, wherein the gas is provided through at least one of the internally formed perforated gussets.

33. The method according to claim 30 or 32, further comprising agitating the culture of phototrophic microorganisms.

34. The method according to claim 33, wherein the culture of phototrophic microorganisms is agitated by supplying the gas through a first internally formed perforated gusset and through a second internally formed perforated gusset on an alternating basis.
